(19) United States
(12) Patent Application Publication
(10) Pub. No.: US 2016/0295085 A1
(43) Pub. Date: Oct. 6, 2016

(54) ENDOSCOPE SYSTEM, PROCESSING APPARATUS AND ENDOSCOPE OPERATING METHOD

(71) Applicant: FUJIFILM Corporation, Tokyo (JP)

(72) Inventor: Tatsuya AOYAMA, Ashigarakami-gun (JP)

(73) Assignee: FUJIFILM Corporation, Tokyo (JP)

(21) Appl. No.: 15/088,983

(22) Filed: Apr. 1, 2016

(30) Foreign Application Priority Data
Apr. 3, 2015 (JP) 2015-077201

(51) Int. Cl. H04N 5/225; A61B 1/00; A61B 1/05; H04N 5/232; A61B 1/06

(52) U.S. Cl. CPC H04N 5/2256; H04N 5/23296; H04N 5/23245; H04N 5/2252; A61B 1/0676; A61B 1/05; A61B 1/00006

(57) ABSTRACT

An endoscope operating method for an endoscope includes a step of applying light to an object through an endoscope tip of the endoscope. The object illuminated with the light is imaged through the endoscope tip. The object is magnified at a first magnification by optical zooming. The object is magnified at a second magnification by electronic zooming according to an image signal obtained by imaging the object. A value relationship between the first and second magnifications is changed according to an illumination type of the light. In case a total magnification for magnifying the object is equal to or less than an upper limit of the first magnification, the object is magnified by the optical zooming. In case the total magnification is more than the upper limit, the object is magnified by the optical zooming and the electronic zooming.

### CROSS-REFERENCE TO RELATED APPLICATION

This application claims priority under 35 USC 119 from Japanese Patent Application No. 2015-077201, filed 3 Apr. 2015, the disclosure of which is incorporated by reference herein.

### BACKGROUND OF THE INVENTION

1. Field of the Invention

The present invention relates to an endoscope system, processing apparatus and endoscope operating method. More particularly, the present invention relates to an endoscope system in which an image of an object can be safely magnified without influence of non-uniformity in irradiation of illuminating light to the object, and processing apparatus and endoscope operating method.

2. Description Related to the Prior Art

An endoscope system is well-known in the field of medical diagnosis, and includes an endoscope, a light source apparatus and a processing apparatus. The endoscope includes an elongated tube for entry in a body cavity of a body of a patient, and creates an image of an object of interest illuminated with light generated by the light source apparatus, for example, mucosa in a gastrointestinal tract. The processing apparatus outputs the image according to a received image signal of an image after imaging the object, and drives a monitor display panel to display the image.

There is an endoscope system in which an object of interest is magnified for imaging by optical zooming and electronic zooming (digital zooming). JP-A 2008-022890 and U.S. Pat. No. 9,030,541 (corresponding to WO 2012/153736) disclose a combined use of the optical zooming and electronic zooming for the magnification operation.

In the optical zooming, a focal length of a lens is adjusted by moving the lens to magnify an image of the object of interest focused on an image sensor, so that an image on a monitor display panel is magnified. In the electronic zooming, a portion of an image of an image signal (for example, center portion) obtained by imaging of the object is trimmed. The trimmed portion is magnified and displayed on the monitor display panel, to magnify the object. The electronic zooming has an advantage of no requirement of a movable part, and advantage of easy magnification operation of the object only by the image processing. However, there is a drawback in lower quality in the image than imaging of the magnification operation by the optical zooming.

In view of the problem, it is conventionally preferable to use the optical zooming with priority, and to use the electronic zooming only after obtaining a maximum magnification of the optical zooming. Such a structure is disclosed in JP-A 2008-022890 and U.S. Pat. No. 9,030,541.

In general, an object to be imaged by the endoscope system is a complete dark state without external light, for example, mucosa in the gastrointestinal tract. Thus, the endoscope system applies illuminating light to the object of interest from the light source apparatus, and forms an image of the object illuminated by the light. It is preferable to consider imaging of the object with light even for magnification operation in the combined use of the optical zooming and electronic zooming. For example, light is applied to an object in an uniform manner. However, non-uniformity in irradiation of the light is likely to give influence upon magnification operation of a portion of the object by the optical zooming, due to a certain value of the magnification for magnification operation. No image light with balanced brightness can be output. This is similar in further magnification operation by electronic zooming for the endoscopic image with influence of the non-uniformity in the irradiation in the light. The non-uniformity of the light is different between various conditions, which are illumination types of the light for imaging, types of the endoscope, and presence and types of a hood device being fitted on an endoscope tip of the endoscope.

### SUMMARY OF THE INVENTION

In view of the foregoing problems, an object of the present invention relates to an endoscope system in which an image of an object can be safely magnified without influence of non-uniformity in irradiation of illuminating light to the object, and processing apparatus and endoscope operating method.

In order to achieve the above and other objects and advantages of this invention, an endoscope system having an endoscope includes a light source for generating light and applying the light to an object through an endoscope tip of the endoscope. An image sensor images the object illuminated with the light through the endoscope tip. A zoom lens system magnifies the object by optical zooming. An electronic zooming device magnifies the object by electronic zooming according to an image signal from the image sensor. A zoom control unit changes a value relationship between a first use amount of the optical zooming and a second use amount of the electronic zooming according to a selected one of an illumination type of the light, a type of the endoscope, and a type of a hood device for fitting on the endoscope tip.

Preferably, the zoom control unit determines an upper limit for the first use amount according to the illumination type, the type of the endoscope, or the type of the hood device.

Preferably, in case a total use amount defined by combining the first and second use amounts is equal to or less than the upper limit, the zoom control unit uses the optical zooming for magnifying the object, and in case the total use amount is more than the upper limit, the zoom control unit uses the optical zooming and the electronic zooming for magnifying the object.

Preferably, the illumination type is constituted by first light and second light being different in a spectral distribution, the first light is used for a first imaging mode, the second light is used for a second imaging mode, and the zoom control unit is operated according to changeover between the first and second imaging modes.

Preferably, the first light is white light, and the second light is light of a short wavelength range which is narrower than a wavelength range of the white light. The zoom control unit sets the upper limit smaller in the second imaging mode than in the first imaging mode.

In one preferred embodiment, the endoscope tip has a lighting window area for exiting the light, and the zoom control unit changes the upper limit according to arrangement of the lighting window area.

Preferably, the zoom control unit sets the upper limit smaller according to smallness in symmetric property of arrangement of the lighting window area with respect to a location of the image sensor.

In another preferred embodiment, the zoom control unit changes the upper limit according to a size of the hood device.

Preferably, the zoom control unit sets the upper limit smaller according to smallness of the size of the hood device.

In still another preferred embodiment, the zoom control unit changes the upper limit according to an angle of an end surface of the hood device relative to the endoscope tip.

Preferably, the zoom control unit sets the upper limit smaller according to greatness of the angle of the end surface.

Preferably, the hood device includes a tubular portion, fitted on a peripheral surface of the endoscope tip, and disposed to extend in an axial direction of the endoscope. The end surface is positioned on a distal end side of the tubular portion, and inclined with reference to the axial direction.

Preferably, the hood device includes a transparent tip cover for covering an end surface of the endoscope tip.

Preferably, each of the illumination type, the type of the endoscope, and the type of the hood device is constituted by first and second types, and non-uniformity of irradiation of the light to the object is larger while the second type is used than while the first type is used. The zoom control unit sets the first use amount smaller while the second type is used than while the first type is used.

Also, a processing apparatus for an endoscope system having an endoscope is provided. The endoscope system includes a light source for generating light and applying the light to an object through an endoscope tip of the endoscope, an image sensor for imaging the object illuminated with the light through the endoscope tip, a zoom lens system for magnifying the object by optical zooming, and an electronic zooming device for magnifying the object by electronic zooming according to an image signal from the image sensor. The processing apparatus includes a zoom control unit for changing a value relationship between a first use amount of the optical zooming and a second use amount of the electronic zooming according to a selected one of an illumination type of the light, a type of the endoscope, and a type of a hood device for fitting on the endoscope tip.

Also, an endoscope operating method for an endoscope includes steps of applying light to an object through an endoscope tip of the endoscope. The object illuminated with the light is imaged through the endoscope tip. The object is magnified by optical zooming. The object is magnified by electronic zooming according to an image signal obtained by imaging the object. A value relationship between a first use amount of the optical zooming and a second use amount of the electronic zooming is changed according to a selected one of an illumination type of the light, a type of the endoscope, and a type of a hood device for fitting on the endoscope tip.

Consequently, an image of an object can be safely magnified without influence of non-uniformity in irradiation of illuminating light to the object, because magnifications of the optical zooming and the electronic zooming are suitably adjusted for obtaining high image quality.

### BRIEF DESCRIPTION OF THE DRAWINGS

The above objects and advantages of the present invention will become more apparent from the following detailed description when read in connection with the accompanying drawings, in which:

FIG. 1 is an explanatory view in a plan, illustrating an endoscope system;

FIG. 2 is a block diagram schematically illustrating the endoscope system;

FIG. 3 is a perspective view illustrating an end surface of an endoscope tip of an endoscope;

FIG. 4 is a graph illustrating a normal image of endoscopy;

FIG. 5 is a graph illustrating the normal image magnified by optical zooming;

FIG. 6 is a graph illustrating the normal image magnified further by electronic zooming;

FIG. 7 is a graph illustrating magnifications of optical zooming and electronic zooming;

FIG. 8 is a graph illustrating magnifications of optical zooming and electronic zooming in a normal imaging mode;

FIG. 9 is a graph illustrating magnifications of optical zooming and electronic zooming in a special imaging mode;

FIG. 10 is a flow chart illustrating operation of zooming;

FIG. 11 is a graph illustrating the zooming in the normal imaging mode;

FIG. 12 is a graph illustrating the zooming in the special imaging mode;

FIG. 13 is a perspective view illustrating another preferred end surface of the endoscope having differently arranged lighting window areas;

FIG. 14 is a perspective view illustrating still another preferred end surface of the endoscope three lighting window areas;

FIG. 15 is a block diagram schematically illustrating an endoscope system of a second preferred embodiment;

FIG. 16 is a graph illustrating magnifications of optical zooming and electronic zooming;

FIG. 17 is a side elevation illustrating a hood device for the endoscope;

FIG. 18 is a block diagram schematically illustrating an endoscope system of a third preferred embodiment;

FIG. 19 is a graph illustrating magnifications of optical zooming and electronic zooming;

FIG. 20 is a flow chart illustrating a table of upper limits and a table of correction ratios;

FIG. 21 is a cross section illustrating a capsule endoscope.

### DETAILED DESCRIPTION OF THE PREFERRED EMBODIMENT(S) OF THE PRESENT INVENTION

### First Embodiment

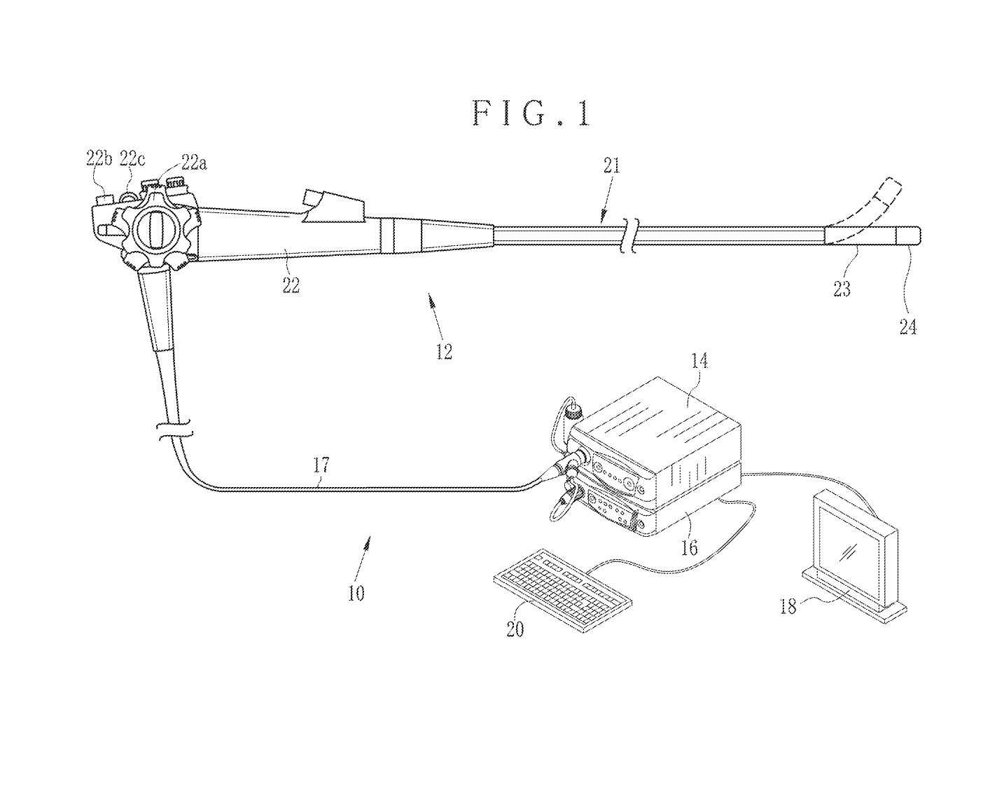

In FIG. 1, an endoscope system 10 includes an endoscope 12, a light source apparatus 14, a processing apparatus 16, a monitor display panel 18, and user terminal equipment 20 or console unit. A universal cable 17 couples the endoscope 12 to the light source apparatus 14 optically, and connects the endoscope 12 to the processing apparatus 16 electrically. The endoscope 12 includes an elongated tube 21 or insertion tube, a control handle housing 22, a steering device 23 and an endoscope tip 24. The elongated tube 21 is entered in a body cavity, for example, a gastrointestinal tract of a body. The control handle housing 22 is disposed at a proximal end of the elongated tube 21. The steering device 23 and the endoscope tip 24 are disposed at a distal end of the elongated tube 21. Steering wheels 22a of the control handle housing 22 are rotated to bend the steering device 23. It is possible to direct the endoscope tip 24 in a desired direction by bending the steering device 23.

A mode switch 22b, a zoom switch 22c, a freeze button (not shown) and the like are disposed on the control handle housing 22 in addition to the steering wheels 22a. Two imaging modes are selectable in the endoscope system 10, inclusive of a normal imaging mode and special imaging mode. In the normal imaging mode, white light of a broad band is applied to an object of interest, which is imaged. In the special imaging mode, narrow band blue light and narrow band green light is applied to an object of interest, which is imaged by enhancing blood vessels, glandular structure and the like, the narrow band blue light and narrow band green light being light of a short wavelength range which is a narrower wavelength range than that of the white light. The normal imaging mode is a first imaging mode of using first light or white light. The special imaging mode is a second imaging mode of using second light, or narrow band blue light and narrow band green light, which is different in a spectral distribution from the white light. The mode switch 22b is used for changing over the normal and special imaging modes. The zoom switch 22c is used for changing a magnification (zoom factor) for imaging an object of interest. The freeze button is used for displaying a still image of the object on the monitor display panel 18.

The processing apparatus 16 is electrically connected with the monitor display panel 18 and the user terminal equipment 20. The monitor display panel 18 displays a normal image, a special image, and relevant information. The normal image is an endoscopic image obtained in a normal imaging mode. The special image is an endoscopic image obtained in a special imaging mode. The user terminal equipment 20 is a user interface for receiving inputs for setting functions or the like. Note that a recorder (not shown) can be connected to the processing apparatus 16 for recording the endoscopic image and image information in association with the endoscopic image.

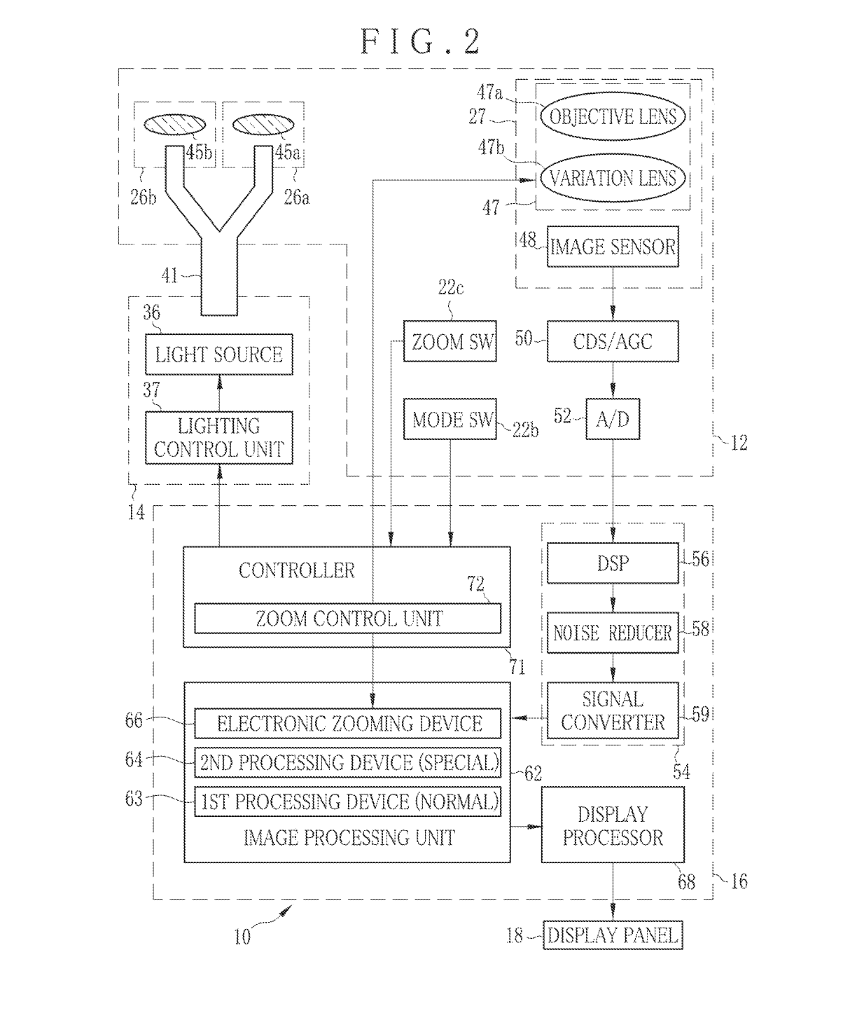

In FIG. 2, the light source apparatus 14 includes a light source 36 and a lighting control unit 37 for controlling the light source 36. An example of the light source 36 includes LEDs (light emitting diodes) of plural colors, and optical filters. The LEDs are violet, blue, green and red LEDs and the like. The optical filters restrict wavelength ranges of light from the LEDs. The lighting control unit 37 controls a time sequence and light amount of the LEDs in the light source 36, movement of the optical filters into and out of a light path, and the like, according to a selected one of the imaging modes. The light source 36 generates white light for illumination in the normal imaging mode, and generates narrow band blue light and narrow band green light in the special imaging mode. Although the light source 36 has the LEDs and optical filters in the embodiment, the light source 36 can be constituted by a light source device of a broad band, such as a xenon lamp and white LED, and optical filters for restricting the wavelength range of the light from the light source device of the broad band. Furthermore, the light source 36 without the LEDs can have a laser diode (LD), phosphor and optical filters. The phosphor generates fluorescence upon receiving laser light from the LD. The optical filters restrict wavelength ranges of the laser light and fluorescence.

Light from the light source 36 is transmitted by a condenser lens, fiber optics, optical coupler and/or the like as optics (not shown), and enters a light guide device 41. The light guide device 41 is contained in the universal cable 17 and the endoscope 12. The light guide device 41 guides the light to the endoscope tip 24 in the endoscope 12. An example of the light guide device 41 can be a multi-mode fiber, such as a fiber cable of a small diameter, having a core diameter of 105 μm, clad diameter of 125 μm, and total diameter of 0.3-0.5 mm in a form inclusive of a protective layer or outer layer.

The endoscope tip 24 of the endoscope 12 has a first lighting window area 26a, a second lighting window area 26b and an imaging unit 27. A lighting lens 45a is disposed with the first lighting window area 26a. Light exited from the light guide device 41 is passed through the lighting lens 45a and applied to the object of interest. Similarly, a lighting lens 45b is disposed with the second lighting window area 26b. Light exited from the light guide device 41 is passed through the lighting lens 45b and applied to the object of interest.

In FIG. 3, the lighting window areas 26a and 26b are arranged in a symmetric manner with respect to the imaging unit 27. Light is emitted equally by the lighting window areas 26a and 26b, of which areas of illumination of the light are different. An end surface 24a or tip surface of the endoscope 12 has the lighting window areas 26a and 26b, which are arranged for illumination of light in a uniform manner to an object of interest. However, non-uniformity in the light may occur. It is likely that non-uniformity of light gives influence to an endoscopic image upon magnifying an object according to the optical zooming. Also, an instrument opening 28 and a nozzle spout of a fluid nozzle 29 are formed in the end surface 24a in addition to the lighting window areas 26a and 26b. The instrument opening 28 is used for protrusion of a medical instrument, such as a forceps. The fluid nozzle 29 ejects fluid, such as water and air.

The imaging unit 27 includes a zoom lens system 47 or optics, and an image sensor 48. The zoom lens system 47 receives object light from an object of interest illuminated by illuminating light and other reflected light components, and focuses the object light on the image sensor 48. The image sensor 48 detects the object light to form an image of the object. The zoom lens system 47 is adjusted to change a magnification by optical zooming, and magnifies or reduces an image of the object of interest. An example of the zoom lens system 47 includes an objective lens 47a and one or more lens elements movable in an optical axis direction of the imaging unit 27. Those lens elements include a variation lens 47b for adjusting a focal length of the imaging unit 27. A zoom control unit 72 is incorporated in a controller 71, and controls the magnification operation and reduction operation for the object of interest in the optical zooming.

The image sensor 48 is a color image sensor and outputs an image signal by imaging of an object. Examples of the image sensor 48 are a CCD image sensor (charge coupled device image sensor), CMOS image sensor (complementary metal oxide semiconductor image sensor) and the like. The image sensor 48 has red, green and blue pixels with red, green and blue color filters arranged on the imaging surface, and outputs an image signal of red, green and blue by photoelectric conversion at the red, green and blue pixels. Note that the image sensor 48 can be a complementary color image sensor with cyan, magenta and yellow color filters as complementary color filters arranged on the imaging surface. For this structure, a color converter can be preferably incorporated in one of the endoscope 12, the light source apparatus 14 and the processing apparatus 16 for color conversion of the image signal of cyan, magenta, yellow and green to the image signal of red, green and blue. It is possible to obtain the image signal of red, green and blue by the color conversion even in the use of the complementary color image sensor.

In FIG. 2, a CDS/AGC device 50 or correlated double sampling/automatic gain control device is supplied with the image signals of the plural colors by the image sensor 48. The CDS/AGC device 50 processes the image signals of the analog form from the image sensor 48 in processing of correlation double sampling (CDS) and automatic gain control (AGC). An A/D converter 52 converts the image signals from the CDS/AGC device 50 into a digital image signal. The image signal is input to the processing apparatus 16.

The processing apparatus 16 includes an image signal acquisition unit 54, an image processing unit 62, a display processor 68 and the controller 71. The image signal acquisition unit 54 acquires an image signal from the endoscope 12. The image signal acquisition unit 54 includes a digital signal processor 56 (DSP), a noise reducer 58 and a signal converter 59.

The digital signal processor 56 processes the image signal for signal processing of various functions, such as defect correction, offset correction, gain correction, linear matrix conversion, gamma conversion, demosaicing, and YC conversion (YC separation). In the defect correction, signals of defective pixels of the image sensor are corrected. In the offset correction, a component of dark current is removed from the image signals after the defect correction, to determine a zero level correctly. In the gain correction, the image signals of red, green and blue after the offset correction are multiplied by a predetermined gain, to adjust a signal level of the image signals. The image signals of the plural colors after the gain correction are processed in the linear matrix conversion for increasing reproductivity of color. After this, gamma conversion is performed to adjust brightness and chromaticity of the image signals. The image signals after the linear matrix conversion are demosaiced in the demosaicing (also referred to as isotropic processing or isochronous processing), to produce signals of color of loss of pixels after interpolation. The demosaicing causes all of the pixels to have signals of red, green and blue. The signal converter 59 processes the demosaiced image signals in the YC conversion, to supply the noise reducer 58 with a luminance signal Y and chrominance signals Cb and Cr.

After demosaicing in the digital signal processor 56, the noise reducer 58 processes the image signals for the noise reduction, for example, according to the movement average method, median filter method or the like. The image signals after the noise reduction are input to the signal converter 59, converted into an image signal of red, green and blue, and input to the image processing unit 62.

The image processing unit 62 includes a first processing device 63 (normal), a second processing device 64 (special) and an electronic zooming device 66.

The first processing device 63 creates a normal image by use of the image signal acquired in the normal imaging mode. The first processing device 63 creates image data of red, green and blue by allocating red, green and blue image signals of one frame to red, green and blue pixels. The first processing device 63 processes the image data of red, green and blue in color conversion, for example, 3×3 matrix processing, gradation conversion, processing of a three-dimensional look-up table, and the like. Then the image data of red, green and blue after the color conversion are processed in color enhancement. The image data of red, green and blue after the color enhancement are processed in structural enhancement, such as enhancement of spacial frequency. The image data of red, green and blue after the structural enhancement are data of the normal image.

The second processing device 64 produces the special image by use of the image signal acquired in the special imaging mode. To this end, the second processing device 64 produces red, green and blue image data by allocating a blue image signal of the narrow band blue light to blue and green pixels, and allocating a green image signal of the narrow band green light to red pixels. Then the second processing device 64 processes the image data for color conversion of various functions, such as matrix processing, gradation conversion and three-dimensional look-up table processing. The second processing device 64 processes the image data after the color conversion for color enhancement and structural enhancement to produce the special image.

The electronic zooming device 66 magnifies an object of interest by electronic zooming with an image signal obtained by imaging of the object. The electronic zooming device 66 partially trims or crops a normal image or special image, and produces a magnified image by magnifying the trimmed portion. A magnification (zoom factor) of the electronic zooming is controlled by the zoom control unit 72. Assuming that the magnification is 1 time, the magnified image is equal to the normal or special image of the original form. The magnified image produced by the electronic zooming device 66 is input to the display processor 68.

Note that pixel interpolation is used in the electronic zooming. A magnification of the magnification operation is set as 2, 3 or 4 times or so or integer times in a limited manner normally, for suppressing noise at the time of the pixel interpolation upon magnifying an object only by the processing of the magnification operation. The electronic zooming device 66 performs the electronic zooming of the total magnification (desired magnification) by combination of the magnification operation of the integer times with a size reduction of a size of the magnified image at a certain magnification, in a condition of suppressing noise in the pixel interpolation. For example, let an object be magnified at 2.4 times. At first, the electronic zooming device 66 magnifies the object at 3 times in the magnification operation, and then reduces the magnified image at a magnification of 0.8 time. Assuming that a total magnification (target magnification) is times of a non-integer, the electronic zooming device 66 magnifies the object at integer times of next higher integer defined by rounding up the non-integer, and then reduces the magnified image at a certain small magnification of a lower number than 1. Furthermore, it is possible in the electronic zooming in the electronic zooming device 66 to set different magnifications between directions, for example, set a magnification of 2 times for a first direction or vertical direction in the image, and a magnification of 3 times for a second direction or horizontal direction.

The display processor 68 converts the magnified image from the electronic zooming device 66 into a display image signal, which is output to the monitor display panel 18. The monitor display panel 18 displays the magnified image formed by magnifying the normal or special image by the electronic zooming.

The controller 71 controls various circuit devices in the endoscope system 10. For example, in case the controller 71 receives a changeover signal of an imaging mode upon operation of the mode switch 22b, the controller 71 controls the lighting control unit 37 to changeover illuminating light between white light for the normal imaging mode, and narrow band blue light and narrow band green light for the special imaging mode.

The controller 71 has the zoom control unit 72. The zoom lens system 47 and the electronic zooming device 66 are controlled by the zoom control unit 72 for magnification operation of an image. A value relationship of the use between (the magnifications of) the optical zooming in the zoom lens system 47 and the electronic zooming in the electronic zooming device 66 is changed.

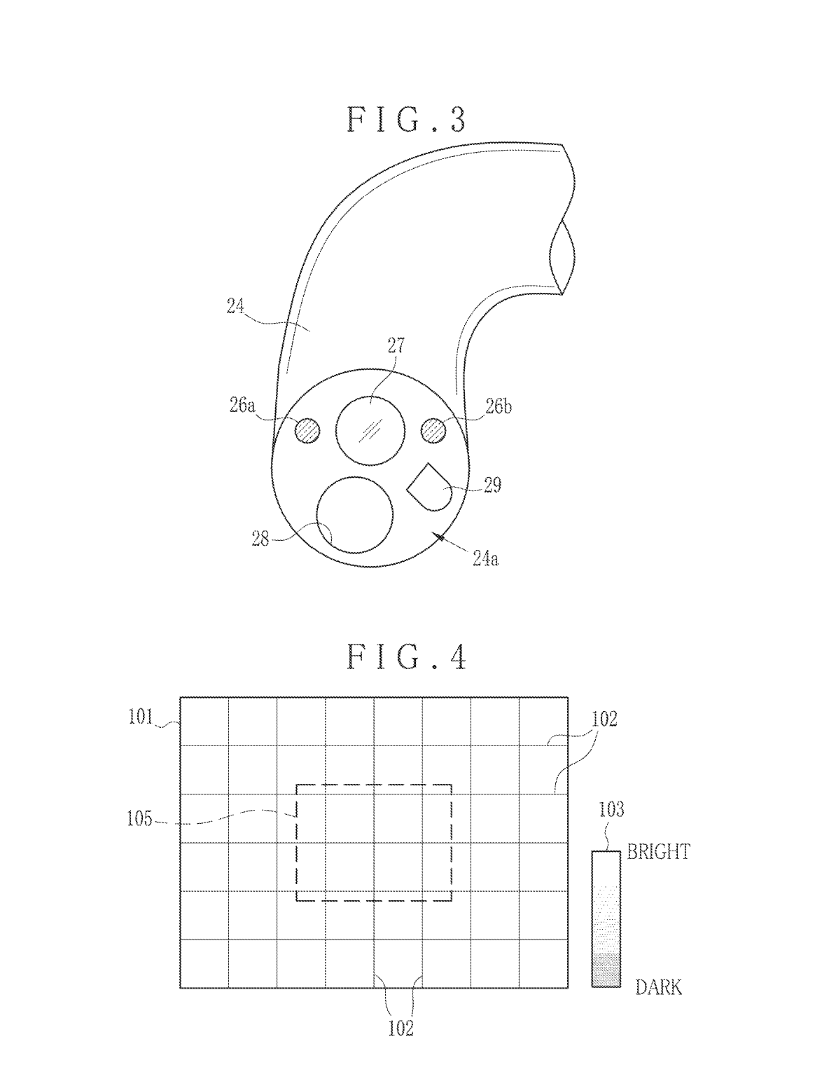

The lighting window areas 26a and 26b in the endoscope 12 emit light to an object of interest. For imaging of the object at a certain distance in the normal imaging mode, white light from the lighting window areas 26a and 26b irradiates the object in a uniform manner. In FIG. 4, a normal image 101 is obtained by imaging the object without zooming. Brightness of the normal image 101 is approximately uniform. Note that a grid pattern 102 is the object of interest in FIG. 4 for clarifying comparison in the magnification in relation to other drawings. A brightness scale 103 is illustrated to express the brightness in light and dim portions. A light portion is expressed in white color. A dim portion is expressed in gray or black color. Those expressions are the same in other drawings.

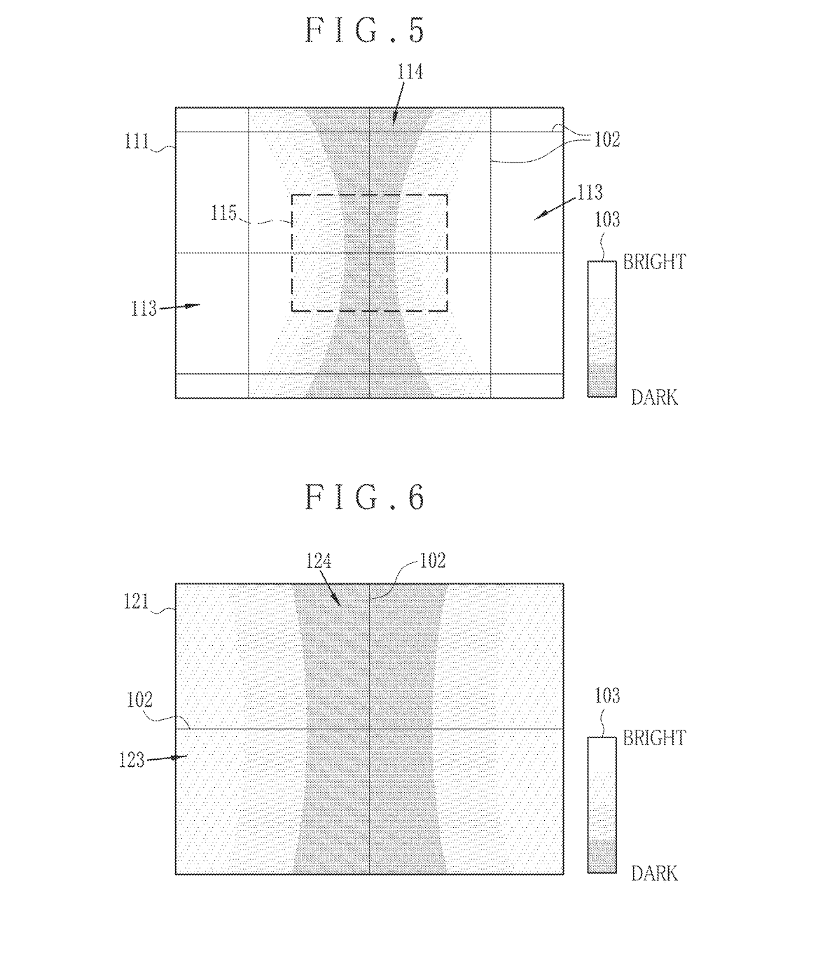

Even assuming that the object of interest is imaged at a certain distance without magnification operation and assuming that brightness of the normal image 101 is approximately uniform on the plane, non-uniformity in the irradiation of light is likely to give influence upon magnifying the object of interest by use of the optical zooming according to a value of the magnification of the object in the optical zooming. Let a region of interest 105 of the broken line be magnified by the optical zooming in FIG. 4. A normal image 111 in FIG. 5 comes to have a bright portion 113 and a dim portion 114. The bright portion 113 and the dim portion 114 correspond to the relative arrangement between the imaging unit 27 and the lighting window areas 26a and 26b, and a relationship between lighting areas of light from the lighting window areas 26a and 26b. Even in case a region of interest 115 of the broken line in FIG. 5 is magnified by the electronic zooming while the bright portion 113 and the dim portion 114 are created, a magnified image 121 after the electronic zooming will have a bright portion 123 and a dim portion 124 as illustrated in FIG. 6 without suitable trimming of the bright portion 113 or the dim portion 114 in FIG. 5 in a selective manner. In the endoscope system 10, an illuminated object is imaged. Assuming that the object is magnified at a critical magnification of the optical zooming, non-uniformity in the irradiation of the light is likely to give influence. Thus, an upper limit of a magnification (zoom factor) of the optical zooming is determined to exit image light with balanced brightness even upon occurrence of the non-uniformity in the irradiation of the light as described above.

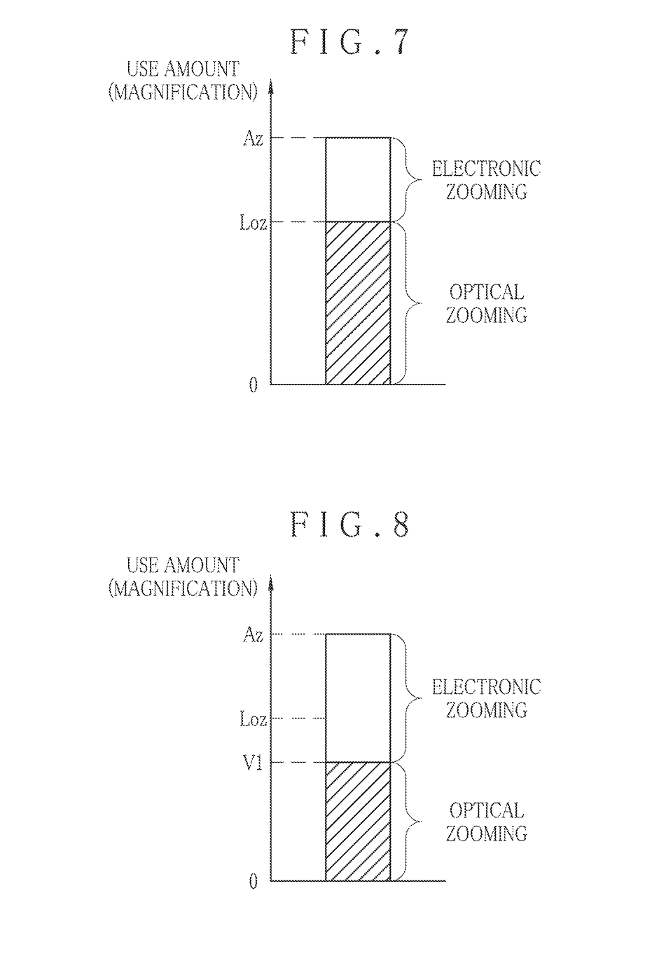

Let Loz be a maximum use amount (information of a magnification) of the optical zooming in association with the movable range of the variation lens 47b. Let Az be a total use amount (information of a total magnification or target magnification) of zooming as a target for a user, and meet a condition of Az>Loz. In FIG. 7, the optical zooming is performed up to the maximum use amount Loz. Then the remaining amount Az-Loz is a use amount of the electronic zooming. The magnification operation of the object up to the maximum use amount Loz by use of the optical zooming may create unbalanced brightness of image light due to non-uniformity in irradiation of the illuminating light.

To solve the problem, the zoom control unit 72 in FIG. 8 determines an upper limit V1 in the normal imaging mode. The upper limit V1 is smaller than the maximum use amount Loz and so determined as to prevent occurrence of unbalanced brightness of image light due to non-uniformity of irradiation of the light, so that the use amount of the optical zooming is limited. Consequently, the zoom control unit 72 reduces the first use amount of the optical zooming in relation to the total use amount Az, to set the second use amount of the electronic zooming equal to or larger than “Az−V1” which is larger than |Az−Loz|.

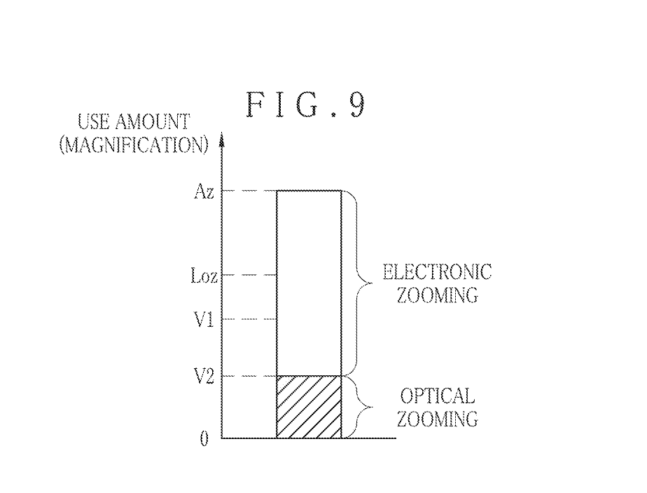

In the special imaging mode, the zoom control unit 72 sets an upper limit V2 which is smaller than the upper limit V1 (V2<V1) for the use amount of the optical zooming. See FIG. 9. The zoom control unit 72 in the special imaging mode sets the use amount of the optical zooming smaller than the use amount in the normal imaging mode in relation to the total use amount Az, so that the use amount of the electronic zooming is increased relatively.

A difference between the upper limits V1 and V2 of the normal and special imaging modes corresponds to a difference of the illumination types of the light in the normal and special imaging modes. The white light in the normal imaging mode includes a long wavelength component which is likely to scatter upon an object of interest and to diffract at the object, for example, a red light component. In contrast, the narrow band blue light and narrow band green light in the special imaging mode is light of a short wavelength range which is a narrower wavelength range than that of the white light. Its scatter upon an object of interest is smaller than the white light, to decrease diffraction at the object. Therefore, non-uniformity of irradiation of light is likely to give greater influence in the special imaging mode than in the normal imaging mode. The zoom control unit 72 determines an upper limit of the optical zooming differently for a selected one of the illumination types of the light or a selected one of the imaging modes.

In the present specification, the use amount of the optical or electronic zooming is information of a magnification. In FIGS. 7 and 8, the total use amount Az (%) (information of the total magnification) of the zooming is indicated as a sum of the first use amount (%) of the optical zooming and the second use amount (%) of the electronic zooming. For expressing the same relationship with the magnification, the total magnification is a product of multiplication between the first magnification of the optical zooming and the second magnification of the electronic zooming. Thus, the second magnification, in case of an unknown value, is acquired by dividing the total magnification by the first magnification.

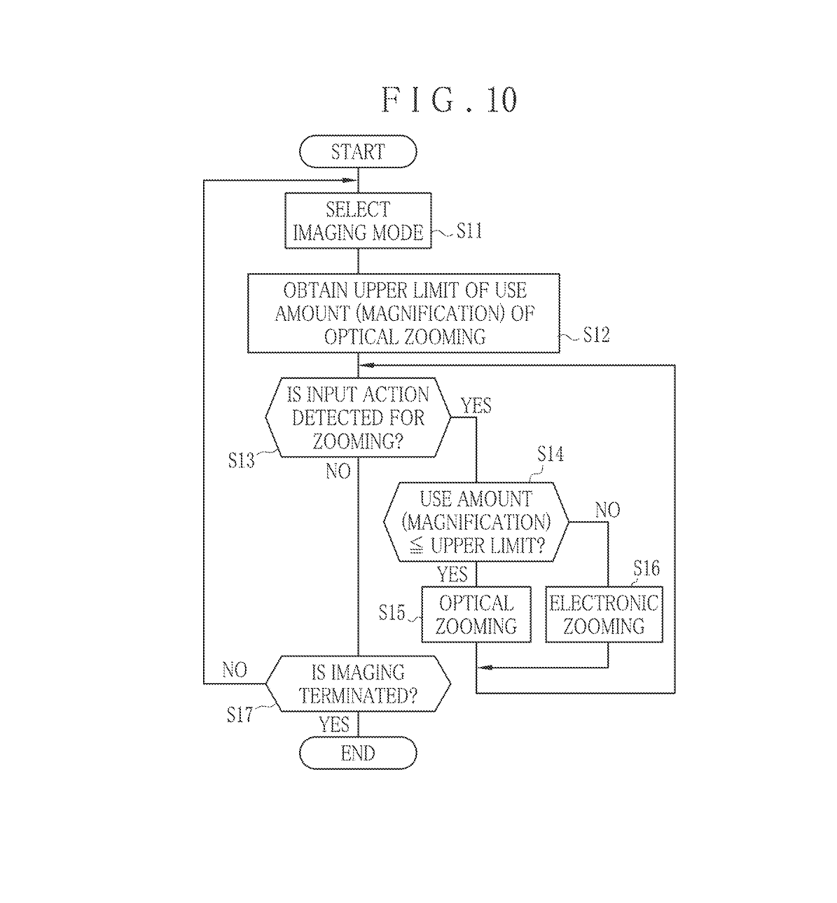

A flow of zoom control in the endoscope system 10 of the embodiment is described by referring to FIG. 10. In case a user operates the mode switch 22b to set an imaging mode in the endoscope system 10 in a step S11, the zoom control unit 72 determines an upper limit for a magnification of the optical zooming in the selected imaging mode in a step S12. An upper limit V1 is set at the time of the normal imaging mode. An upper limit V2 smaller than the upper limit V1 is set at the time of the special imaging mode. Then the zoom control unit 72 monitors a state of the zoom switch 22c.

An input action to the zoom switch 22c is detected (yes in a step S13). Then the zoom control unit 72 compares a (total) magnification of an object of interest at the time of the input action of the zoom switch 22c with the upper limit of the magnification of the optical zooming in a step S14. Assuming that the (total) magnification of the object of interest is equal to or less than the upper limit of the magnification of the optical zooming, the zoom control unit 72 controls the zoom lens system 47 to magnify the object by optical zooming in a step S15. Assuming that the (total) magnification of the object at the time of the input action of the zoom switch 22c is more than the upper limit of the magnification of the optical zooming, then the electronic zooming device 66 is controlled to magnify the object of interest by the electronic zooming in a step S16.

In FIG. 11, the zoom switch 22c is operated continuously by a user while the imaging mode is the normal imaging mode. An object of interest becomes magnified by the optical zooming from a start time point (time 0) of the zooming until time T1 of the reach of the magnification to the upper limit V1 of the normal imaging mode. Then the zoom switch 22c continues being operated after the time T1. The object is magnified by the electronic zooming while the magnification of the optical zooming is kept at the upper limit V1.

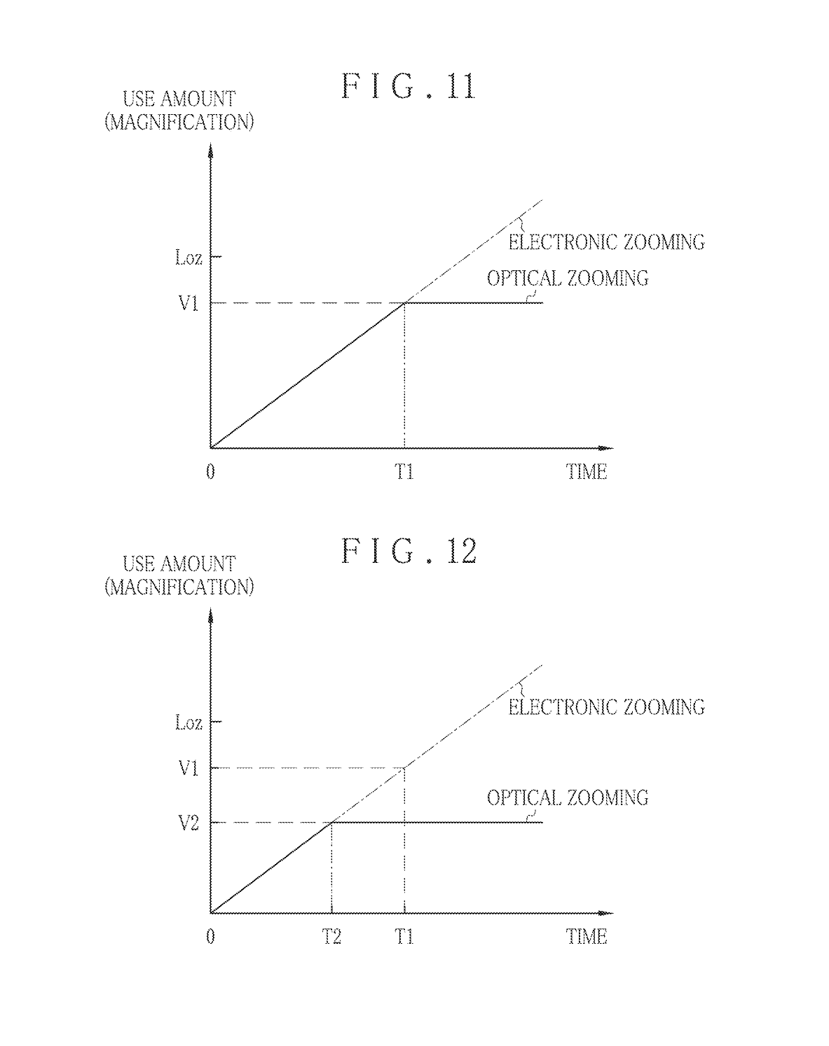

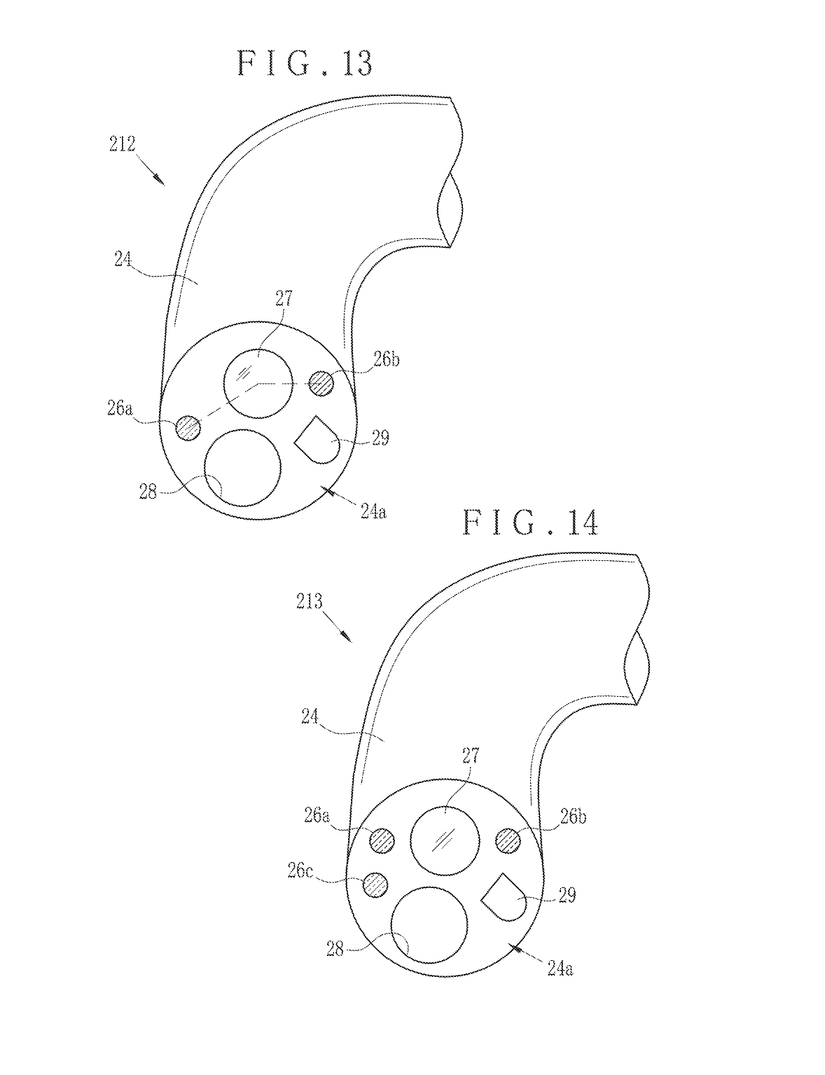

In FIG. 12, the imaging mode is the special imaging mode. In case a continuous input action of the zoom switch 22c is performed by the user, the object is magnified by optical zooming from a start time point of the zooming (time 0) until time T2 of a reach of the magnification to the upper limit V2 for the special imaging mode. Then the object is magnified by the electronic zooming. Non-uniformity in irradiation of light is likely to give influence in the optical zooming due to small diffraction of the light in comparison with operation in the normal imaging mode. As the upper limit V2 for the special imaging mode is set smaller than the upper limit V1 for the normal imaging mode, time required for a reach of the magnification to the upper limit V2 in the optical zooming is shorter in the special imaging mode than in the normal imaging mode. In short, the use amount of the optical zooming is lower in the special imaging mode than in the normal imaging mode upon magnifying the object over the upper limit V2 of the magnification of the optical zooming. The use amount of the electronic zooming becomes higher. The endoscope system 10 performs the zooming control repeatedly until the end of the imaging. See a step S17 in the drawing.

In the endoscope system 10, the value relationship of the use between (the magnifications of) the optical zooming and the electronic zooming is changed by determining the upper limit of the magnification for an object in the optical zooming so as to prevent occurrence of unbalanced brightness of image light due to non-uniformity of the irradiation. Thus, it is possible in the endoscope system 10 to magnify the object up to a total magnification (desired magnification) to prevent influence of non-uniformity in an endoscopic image after the irradiation of the light.

Assuming that the magnification of the object is equal to or lower than the upper limit of the magnification of the object in the optical zooming, the endoscope system 10 magnifies the object by the optical zooming. Incase the magnification becomes more than the upper limit of the magnification of the optical zooming, the endoscope system 10 further magnifies the object by the electronic zooming. It is possible to display an endoscopic image on the monitor display panel 18 with minimized noise in a range without unbalanced brightness of image light due to non-uniformity of irradiation of the light.

Also, the endoscope system 10 sets an upper limit of the magnification of the optical zooming according to an illumination type of illuminating light. In the special imaging mode for use of light of a short wavelength range and of which the wavelength range is narrower than that of the white light, the upper limit V2 of the optical zooming is set smaller than the upper limit V1 of the normal imaging mode with the white light. Thus, the endoscope system 10 changes an upper limit of the optical zooming and sets the upper limit suitable for the illumination type of the illuminating light by considering risk of non-uniformity in the white light for the normal imaging mode and narrow band blue light and narrow band green light for the special imaging mode. It is possible to magnify the object up to a total magnification to exit image light with balanced brightness even upon occurrence of non-uniformity in the irradiation of the light after changing over the imaging mode and after changes in property of the illuminating light.

### Second Embodiment

In the first embodiment, the illumination type of the illuminating light is considered for adjustment of the use of the optical and electronic zooming. However, it is possible to consider a type of an endoscope for adjustment of the use of the optical and electronic zooming.

In the first embodiment, the lighting window areas 26a and 26b are symmetric with one another in the end surface 24a. In contrast, an endoscope 212 in FIG. 13 has the lighting window areas 26a and 26b arranged asymmetrically in the end surface 24a with reference to the imaging unit 27. In FIG. 14, another preferred endoscope 213 includes a third lighting window area 26c in addition to the lighting window areas 26a and 26b. The third lighting window area 26c is constructed equally with the lighting window areas 26a and 26b. The lighting window areas 26a, 26b and 26c are asymmetric with reference to the imaging unit 27.

In the endoscope 212 or 213 having low symmetric property in arrangement of lighting window areas for exiting light with respect to the imaging unit 27, non-uniformity of irradiation of light is likely to give greater influence upon the optical zooming than in the endoscope 12 of the first embodiment in which the symmetric property in arrangement of lighting window areas is high with respect to the imaging unit 27. The number of the lighting window areas and symmetric property of the arrangement of the lighting window areas with respect to the imaging unit 27 are different between types of the endoscope. It is preferable to detect the type of the endoscope and change the value relationship of the use between the optical zooming and electronic zooming according to the type of the endoscope. Note that the symmetric property of the arrangement of the lighting window areas with respect to the imaging unit 27 is expressed by degree of coincidence between a location of the imaging unit 27 (or the center of the imaging unit 27) and a gravity center of the lighting window areas. The disposition of the lighting window areas with the highest symmetric property is the disposition of coincidence of the gravity center of the lighting window areas with the imaging unit 27. The symmetric property of the lighting window areas decreases according to an increase in a distance between the gravity center of the lighting window areas and the imaging unit 27. Assuming that the lighting window areas are two, their gravity center is a midpoint between those.

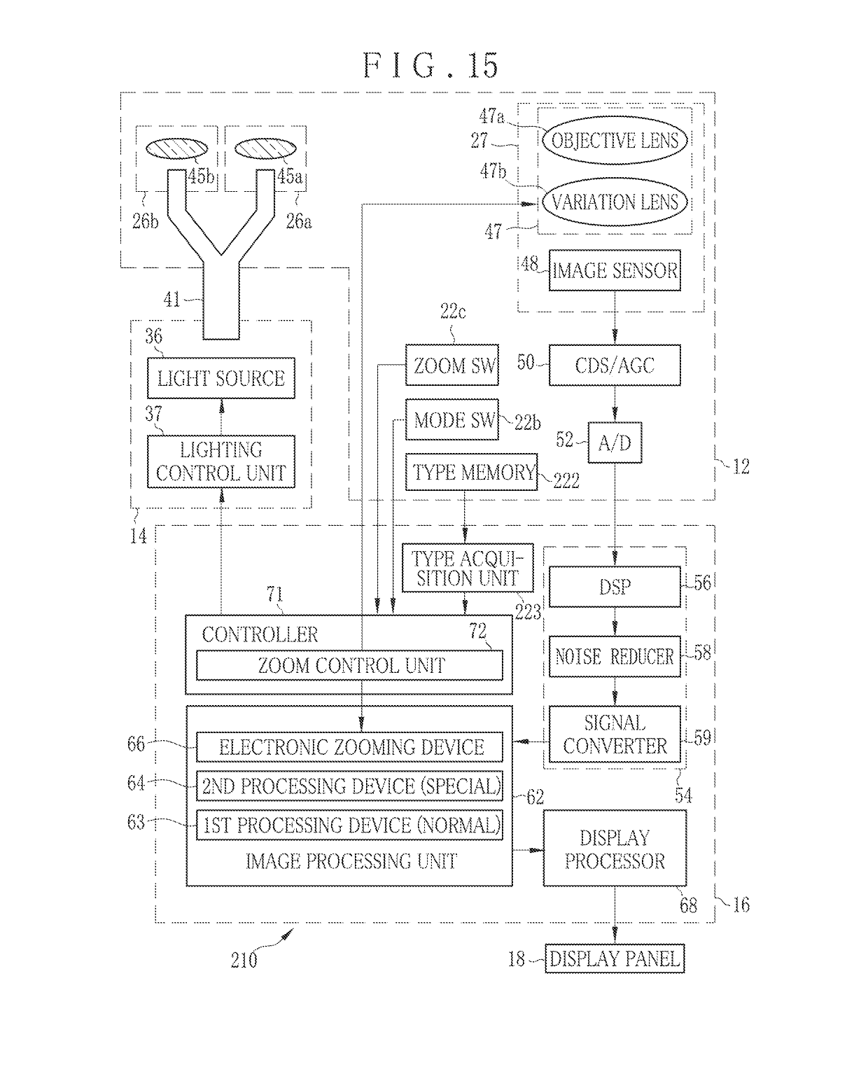

It is preferable to determine an upper limit of the magnification of the optical zooming according to types of the endoscope. For example, an endoscope system 210 in FIG. 15 includes a type memory 222 or type storage medium incorporated in the endoscope 12 for storing type information related to the number of lighting window areas, their arrangement relative to the imaging unit 27, and the like. In the endoscope system 210, a type acquisition unit 223 is incorporated in the processing apparatus 16, and reads the type information from the type memory 222 in response to connection of the endoscope 12 to the processing apparatus 16, and inputs the type information to the zoom control unit 72. The zoom control unit 72 determines an upper limit of the magnification (zoom factor) of the object of interest according to optical zooming by use of the type information of the endoscope, so as to change the value relationship of the use between the optical zooming and the electronic zooming according to the type of the endoscope 12.

In FIG. 16, the endoscope 12 is the type A1. The zoom control unit 72 sets the magnification of the optical zooming at an upper limit VA1. Assuming that the endoscope 12 is a type A2 different from the type A1, and assuming that non-uniformity in irradiation of light upon optical zooming does not give remarkable influence because of higher symmetric property of the lighting window areas with respect to the imaging unit 27 than that in the type A1, then an upper limit VA2 for the magnification of the optical zooming is set at a higher level than that for the type A1. Assuming that the endoscope 12 is a type A3 different from the type A1, and assuming that non-uniformity in irradiation of light upon optical zooming gives influence with high possibility because of lower symmetric property of the lighting window areas with respect to the imaging unit 27 than that in the type A1, then an upper limit VA3 for the magnification of the optical zooming is set at a lower level than that for the type A1.

In the endoscope system 210 of the second embodiment, the value relationship of the use between (the magnifications of) the optical zooming and the electronic zooming is changed according to a type of the endoscope 12. It is possible to magnify an object to a total magnification (desired magnification) without exiting image light with unbalanced brightness due to non-uniformity of irradiation of light, no matter which of the types of the endoscope 12 is used.

### Third Embodiment

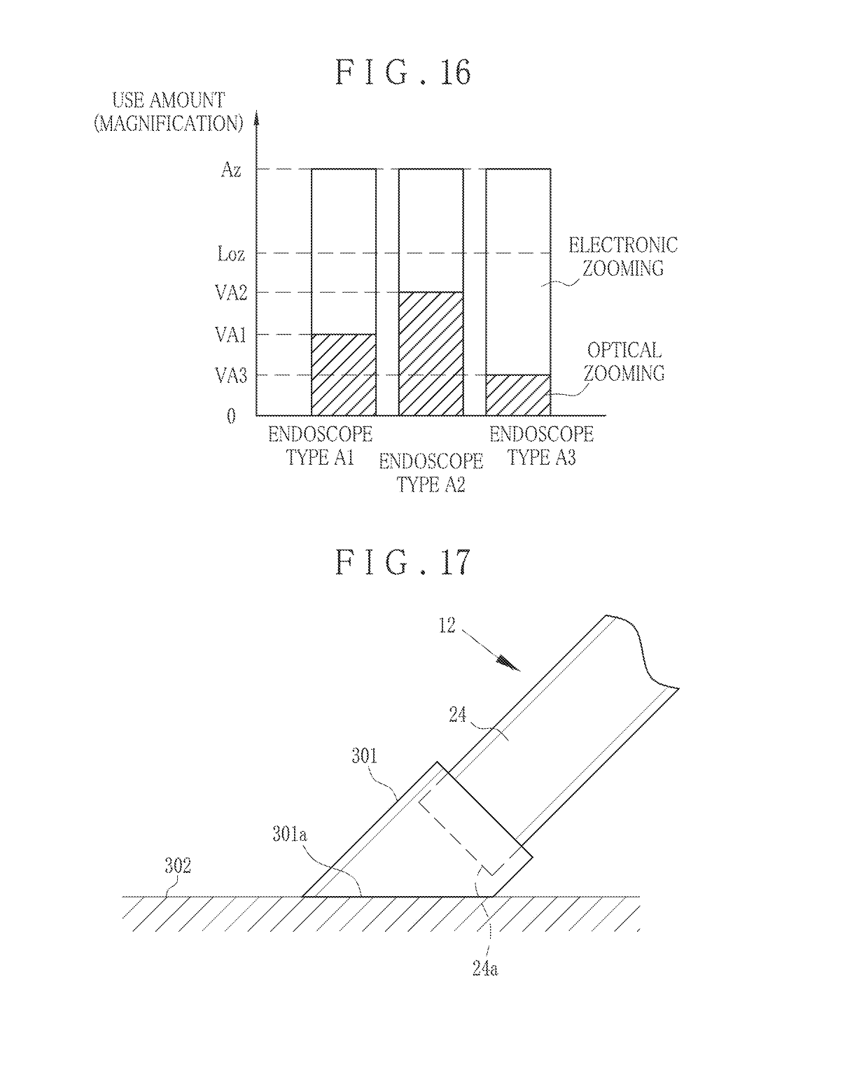

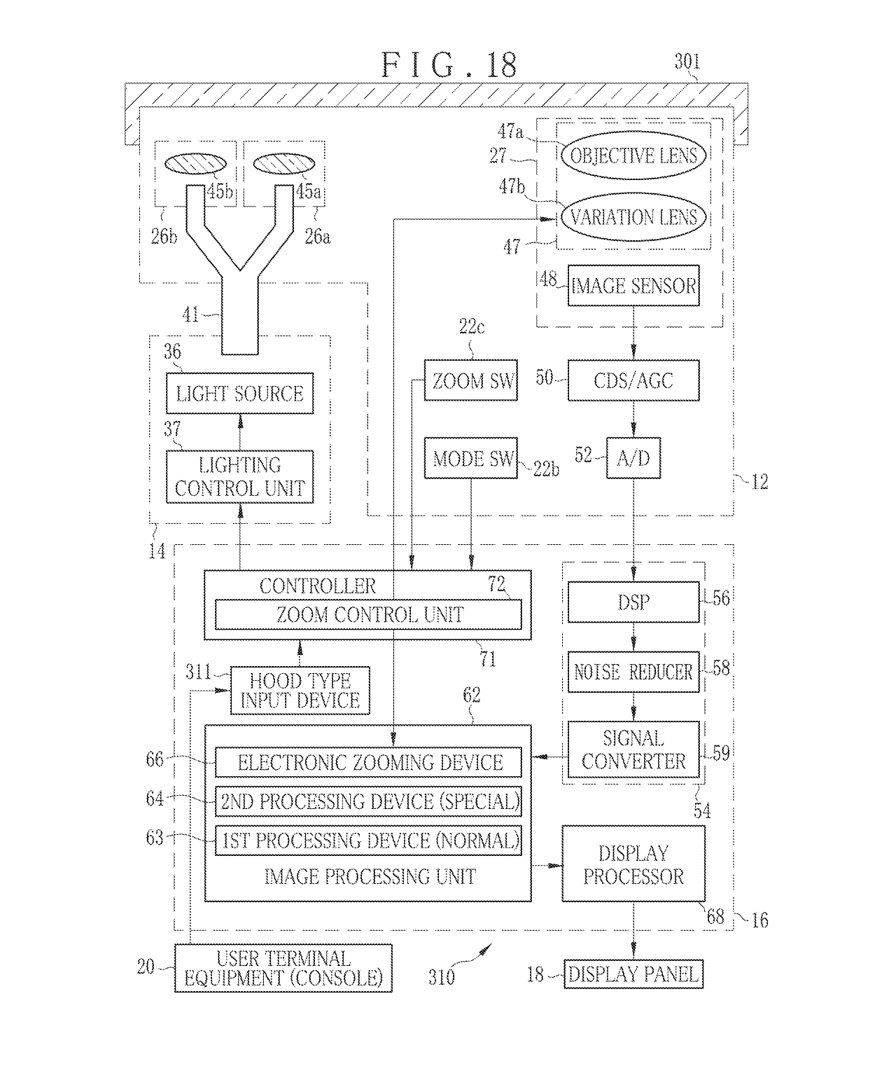

In the first and second embodiments, the endoscope tip 24 of the endoscope 12 is used directly to diagnose an object of interest in the body cavity. In contrast, a hood device 301 or tip cover or tip protector (or cap) is fitted on the endoscope tip 24 of the endoscope 12 for use, as illustrated in FIG. 17. An end surface 301a or tip surface of a tubular portion of the hood device 301 on the endoscope tip 24 is set in contact with an object of interest 302, so as to keep the end surface 24a of the endoscope 12 oriented stably with respect to the object of interest 302 in relation to a distance, angle and the like. There are plural types of the hood device 301 with differences in an angle, distance and the like of the end surface 301a in relation to the end surface 24a of the endoscope 12. Influence of non-uniformity in irradiation of light in the optical zooming differs between various conditions of the light relative to the object of interest, for example, angle, distance and the like of the light. Thus, it is preferable to change the value relationship of the use between the optical zooming and electronic zooming according to the types of the hood device 301.

In FIG. 18, a hood type input device 311 (interface port) is incorporated in the processing apparatus 16 in an endoscope system 310 to set an upper limit for the magnification of the optical zooming according to the type of the hood device 301. The user terminal equipment 20 is manipulated to select a type of the hood device 301 for use in connection with the hood type input device 311. The hood type input device 311 inputs information of the type of the hood device 301 being selected to the zoom control unit 72. The zoom control unit 72 determines the upper limit of the magnification of the optical zooming according to the type of the hood device 301, to change a value relationship of the use between the optical zooming and the electronic zooming.

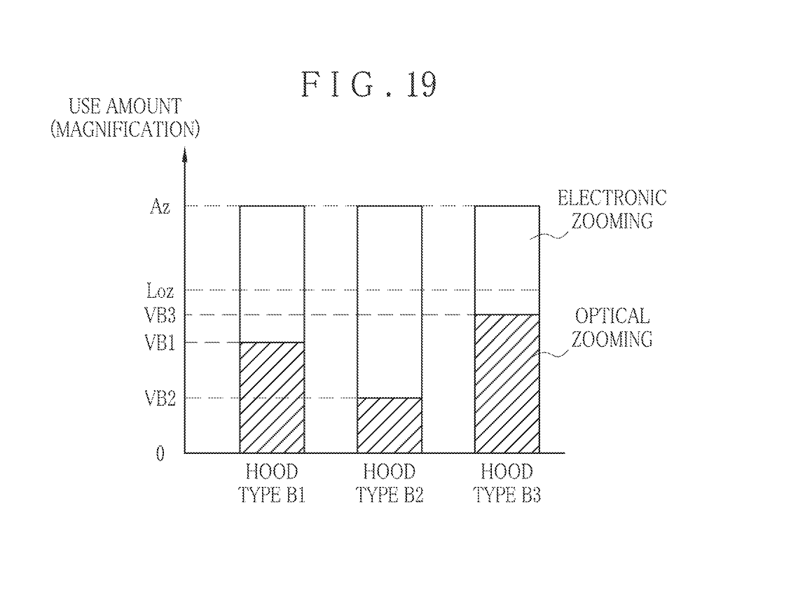

In FIG. 19, the hood device 301 is the type B1. The zoom control unit 72 sets the magnification of the optical zooming at an upper limit VB1. Assuming that the hood device 301 is a type B2 different from the type B1, and assuming that non-uniformity in irradiation of light upon optical zooming gives influence with high possibility, then an upper limit VB2 for the magnification of the optical zooming is set at a lower level than that according to the type B1. Assuming that the hood device 301 is a type B3 different from the type B1, and assuming that non-uniformity in irradiation of light upon optical zooming does not give remarkable influence, then an upper limit VB3 for the magnification of the optical zooming is set at a higher level than that according to the type B1.

In the endoscope system 310 of the third embodiment, the value relationship of the use between (the magnifications of) the optical zooming and the electronic zooming is changed according to a type of the hood device 301. It is possible to magnify an object to a total magnification without exiting image light with unbalanced brightness due to non-uniformity of irradiation of light, no matter which of the types of the hood device 301 is used.

In the third embodiment, the size of the hood device 301 (tubular portion) and the angle of the end surface 301a of the hood device 301 relative to the end surface 24a of the endoscope 12 are considered in combination, to determine an upper limit of the magnification in the optical zooming. Furthermore, an upper limit of the magnification in the optical zooming can be preferably decreased according to smallness of the hood device 301 (tubular portion). A distance from the end surface 24a to the object of interest 302 is decreased according to a decrease in the size of the hood device 301, to create risk of occurrence of non-uniformity in irradiation of light due to the arrangement of the lighting window areas 26a and 26b. However, assuming that the size of the hood device 301 is small, a state of being out of focus may occur without performing optical zooming in a large manner according to a relationship with a focal length. For this structure, an upper limit of the magnification of the optical zooming is determined in an in-focus condition.

The upper limit of the magnification in the optical zooming can be determined according to the angle of the end surface 301a of the hood device 301. To this end, it is preferable to set the upper limit of the magnification in the optical zooming at a small value according to greatness of the angle of the end surface 301a of the hood device 301 relative to the end surface 24a of the endoscope 12, namely, nearness of an angle difference to 90 degrees between the end surface 301a and the end surface 24a. Light can be applied at an angle near to the parallel form relative to the object of interest 302 according to greatness in the angle difference between the end surface 301a of the hood device 301 and the end surface 24a of the endoscope 12. There is risk of non-uniformity of irradiation of the light in a form of increasing brightness according to nearness to the end surface 24a of the endoscope 12 and decreasing brightness according to the distance from the end surface 24a of the endoscope 12.

The value relationship of the use between (the magnifications of) the optical zooming and the electronic zooming is changed according to the illumination type of the light or imaging mode in the first embodiment, according to the type of the endoscope 12 in the second embodiment, and according to the type of the hood device 301 in the third embodiment. However, two or more of the illumination type of the light, the type of the endoscope 12 and the type of the hood device 301 can be selectively combined and considered for the purpose of determining an upper limit of the magnification in the optical zooming, so that the value relationship of the use between the optical zooming and the electronic zooming can be changed. Furthermore, it is possible to utilize plural values of the upper limits and select one of the values for an available upper limit. For example, a smallest upper limit is selected among a first upper limit determined from the illumination type of the light in the first embodiment, a second upper limit determined from the type of the endoscope 12 in the second embodiment, and a third upper limit determined from the type of the hood device 301 in the third embodiment. The smallest upper limit can be set for the available upper limit for the magnification in the optical zooming.

Furthermore, a reference endoscope can be predetermined as a reference type of endoscope. Values of upper limits of a magnification according to the optical zooming in the use of the reference endoscope are predetermined for respective illumination types of the illuminating light and respective types of the hood devices. In relation to various endoscopes other than the reference endoscope, it is unnecessary to predetermine numerous upper limits of the magnification according to the optical zooming for the illumination types of the light and the types of the hood devices. Upper limits of the magnification according to the optical zooming can be calculated only by arithmetic processing according to the reference endoscope.

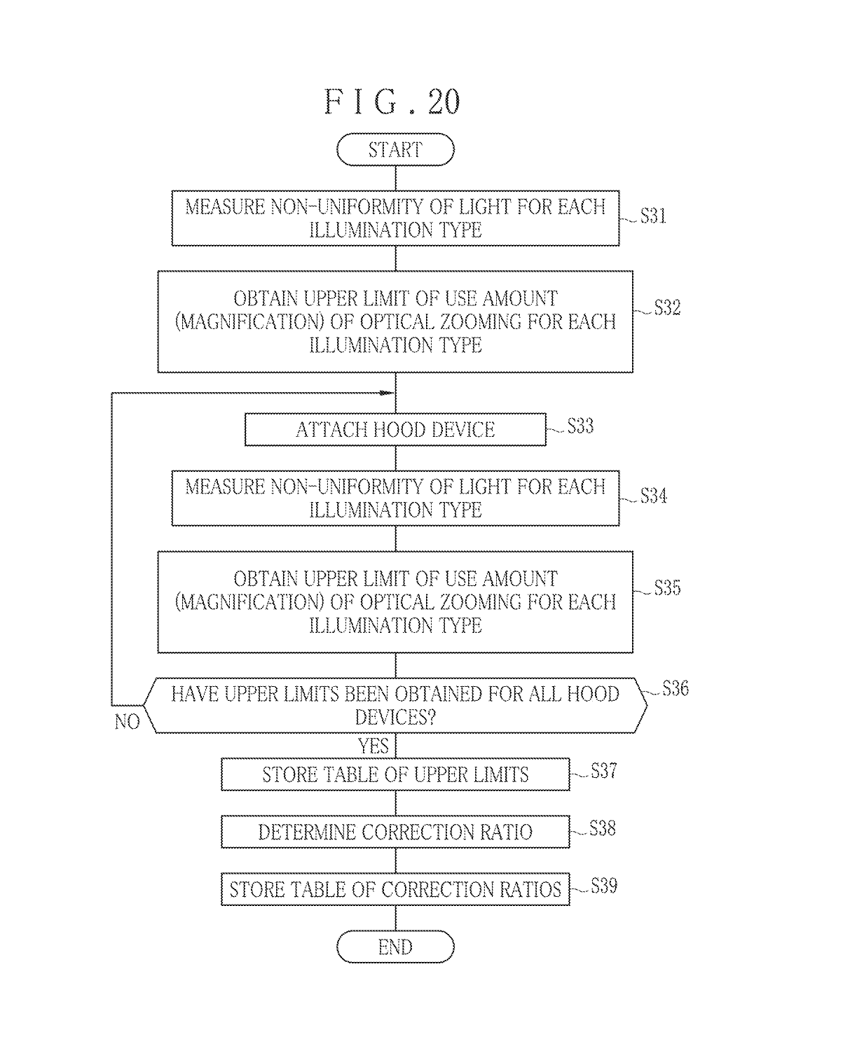

In FIG. 20, the reference endoscope without the hood device 301 is used, to measure non-uniformity in irradiation of the light for each of the illumination types of the light in a step S31. An upper limit of the magnification in the optical zooming is determined for each illumination type of the light in the use of the reference endoscope by use of the results of the measurement, in a step S32. Specifically, the upper limit V1 of the magnification of the optical zooming for imaging in the normal imaging mode by use of the reference endoscope is determined. Also, the upper limit V2 of the magnification of the optical zooming for imaging in the special imaging mode by use of the reference endoscope is determined.

Then the hood device 301 is mounted on a reference endoscope in a step S33, to measure non-uniformity in the irradiation of the light for the respective illumination types of the light in a step S34. An upper limit of the magnification of the optical zooming in combination with the hood device 301 is determined for each illumination type of the light by use of a result of the measurement in a step S35. Also, the hood device 301 is replaced with a second hood device (tip cover), so that an upper limit of the magnification of the optical zooming in combination with the hood device 301 is determined for each illumination type of the light in the same manner as the above in relation to the hood devices of the all the types compatible with the endoscope system 10 in a step S36. Thus, an upper limit of the magnification of the optical zooming in the use of the reference endoscope is determined for each illumination type of the light and each status and type of the hood device (inclusive of lack of a hood device). Information of the upper limit is stored in a form of an upper limit table, which is stored in a storage medium in a step S37. Also, an upper limit of the magnification of the optical zooming is compared between structures with and without a hood device. A ratio of correction for correcting an upper limit of the magnification of the optical zooming of the structure with the hood device is determined in relation to the structure without the hood device, in a step S38. The ratio of the correction is obtained for each of the illumination types of the light and for all of the hood devices. The zoom control unit 72 is caused to store a correction ratio table of information of the ratios of the correction in a step S39.

As the zoom control unit 72 stores the upper limit table described above, the upper limit of the magnification of the optical zooming on a condition of using a certain endoscope other than the reference endoscope and not using a hood device can be determined by use of the upper limit table and the type information of the endoscope for use. Also, it is possible in a condition with a hood device to determine the upper limit of the magnification of the optical zooming without use of the hood device by correction according to the correction ratio table.

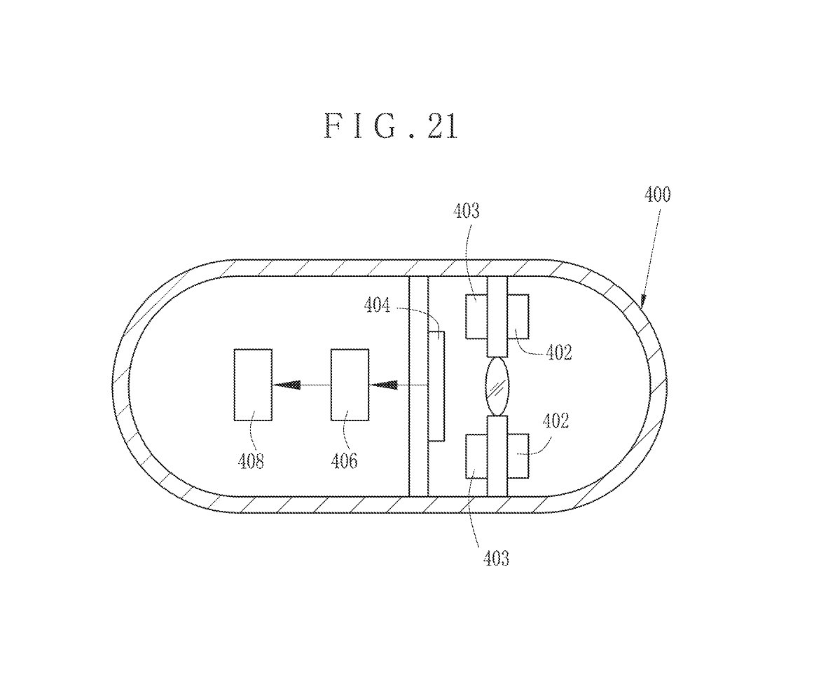

In the first, second and third embodiments, the endoscope 12 is a normal type which is entered to the body cavity in its axial direction. In contrast, a capsule endoscope 400 of FIG. 21 can be used in the present invention. A capsule endoscope system includes the capsule endoscope 400 swallowable in a patient body, and a processing apparatus (not shown).

The capsule endoscope 400 includes a light source 402, a controller 403, an image sensor 404, an image generator 406 (processor) and a communication antenna 408 or wireless antenna. The light source 402 corresponds to the light source 36 in the above embodiments.

The controller 403 operates in the same manner as the lighting control unit 37 and the controller 71 (the zoom control unit 72) in the above embodiments. The controller 403 is communicable with a processing apparatus included in the capsule endoscope system wirelessly by use of the communication antenna 408. The processing apparatus herein is basically the same as the processing apparatus 16 of the above embodiments and variants. The image generator 406 is contained in the capsule endoscope 400, and an endoscope image is transmitted by the communication antenna 408 to the processing apparatus. The image sensor 404 is constructed in the same structure as the image sensor 48 of the above embodiments.

The electronic zooming device 66 in the first to third embodiments performs the electronic zooming for magnification operation of any total magnification (desired magnification), which is freely determined according to the illumination type of the light, the type of the endoscope, and type of the hood device. However, the value of the magnification of the electronic zooming should be set at integer times in order to suppress noise. Thus, it is preferable to set an upper limit of the magnification of the optical zooming equal to a product of multiplying a “reciprocal number of an integer” by the maximum magnification of the optical zooming according to the illumination type of the light, the type of the endoscope, and type of the hood device. Consequently the magnification can be corrected in the electronic zooming of integer times for the magnification without shortage or surplus because the upper limit of the magnification of the optical zooming can be suitably determined.

In a preferred embodiment mode of the invention, an endoscope system having an endoscope includes a light source for generating light and applying the light to an object through an endoscope tip of the endoscope. An image sensor images the object illuminated with the light through the endoscope tip. A zoom lens system magnifies the object at a first magnification by optical zooming. An electronic zooming device magnifies the object at a second magnification by electronic zooming according to an image signal from the image sensor. A zoom control unit changes a value relationship between the first and second magnifications according to a selected one of an illumination type of the light, a type of the endoscope, and a type of a hood device for fitting on the endoscope tip.

Preferably, the zoom control unit determines an upper limit for the first magnification according to the illumination type, the type of the endoscope, or the type of the hood device.

Although the present invention has been fully described by way of the preferred embodiments thereof with reference to the accompanying drawings, various changes and modifications will be apparent to those having skill in this field. Therefore, unless otherwise these changes and modifications depart from the scope of the present invention, they should be construed as included therein.

What is claimed is:

1. An endoscope system having an endoscope, comprising:
alight source for generating light and applying said light to an object through an endoscope tip of said endoscope;
an image sensor for imaging said object illuminated with said light through said endoscope tip;
a zoom lens system for magnifying said object by optical zooming;
an electronic zooming device for magnifying said object by electronic zooming according to an image signal from said image sensor;
a zoom control unit for changing a value relationship between a first use amount of said optical zooming and a second use amount of said electronic zooming according to a selected one of an illumination type of said light, a type of said endoscope, and a type of a hood device for fitting on said endoscope tip.

2. An endoscope system as defined in claim 1, wherein said zoom control unit determines an upper limit for said first use amount according to said illumination type, said type of said endoscope, or said type of said hood device.

3. An endoscope system as defined in claim 2, wherein in case a total use amount defined by combining said first and second use amounts is equal to or less than said upper limit, said zoom control unit uses said optical zooming for magnifying said object, and in case said total use amount is more than said upper limit, said zoom control unit uses said optical zooming and said electronic zooming for magnifying said object.

4. An endoscope system as defined in claim 2, wherein said illumination type is constituted by first light and second light being different in a spectral distribution, said first light is used for a first imaging mode, said second light is used for a second imaging mode, and said zoom control unit is operated according to changeover between said first and second imaging modes.

5. An endoscope system as defined in claim 4, wherein said first light is white light, and said second light is light of a short wavelength range which is narrower than a wavelength range of said white light;
said zoom control unit sets said upper limit smaller in said second imaging mode than in said first imaging mode.

6. An endoscope system as defined in claim 2, wherein said endoscope tip has a lighting window area for exiting said light, and said zoom control unit changes said upper limit according to arrangement of said lighting window area.

7. An endoscope system as defined in claim 6, wherein said zoom control unit sets said upper limit smaller according to smallness in symmetric property of arrangement of said lighting window area with respect to a location of said image sensor.

8. An endoscope system as defined in claim 2, wherein said zoom control unit changes said upper limit according to a size of said hood device.

9. An endoscope system as defined in claim 8, wherein said zoom control unit sets said upper limit smaller according to smallness of said size of said hood device.

10. An endoscope system as defined in claim 2, wherein said zoom control unit changes said upper limit according to an angle of an end surface of said hood device relative to said endoscope tip.

11. An endoscope system as defined in claim 10, wherein said zoom control unit sets said upper limit smaller according to greatness of said angle of said end surface.

12. An endoscope system as defined in claim 10, wherein said hood device includes a tubular portion, fitted on a peripheral surface of said endoscope tip, and disposed to extend in an axial direction of said endoscope;
said end surface is positioned on a distal end side of said tubular portion, and inclined with reference to said axial direction.

13. An endoscope system as defined in claim 1, wherein said hood device includes a transparent tip cover for covering an end surface of said endoscope tip.

14. An endoscope system as defined in claim 1, wherein each of said illumination type, said type of said endoscope, and said type of said hood device is constituted by first and second types, and non-uniformity of irradiation of said light to said object is larger while said second type is used than while said first type is used;
said zoom control unit sets said first use amount smaller while said second type is used than while said first type is used.

15. A processing apparatus for an endoscope system having an endoscope, said endoscope system including a light source for generating light and applying said light to an object through an endoscope tip of said endoscope, an image sensor for imaging said object illuminated with said light through said endoscope tip, a zoom lens system for magnifying said object by optical zooming, and an electronic zooming device for magnifying said object by electronic zooming according to an image signal from said image sensor, said processing apparatus comprising:
a zoom control unit for changing a value relationship between a first use amount of said optical zooming and a second use amount of said electronic zooming according to a selected one of an illumination type of said light, a type of said endoscope, and a type of a hood device for fitting on said endoscope tip.

16. An endoscope operating method for an endoscope, comprising steps of:
applying light to an object through an endoscope tip of said endoscope;
imaging said object illuminated with said light through said endoscope tip;
magnifying said object by optical zooming;
magnifying said object by electronic zooming according to an image signal obtained by imaging said object;
changing a value relationship between a first use amount of said optical zooming and a second use amount of said electronic zooming according to a selected one of an illumination type of said light, a type of said endoscope, and a type of a hood device for fitting on said endoscope tip.
